(19) United States
(12) Patent Application Publication
(10) Pub. No.: US 2015/0225687 A1
(43) Pub. Date: Aug. 13, 2015

(54) Systems for Making Host Cells with Artificial Endosymbionts

(71) Applicant: BELL BIOSYSTEMS, INC., Palo Alto, CA (US)

(72) Inventors: Caleb Bell, III, San Mateo, CA (US); Alexey Bazarov, Fremont, CA (US); Brad Rice, Daville, CA (US)

(21) Appl. No.: 14/620,691

(22) Filed: Feb. 12, 2015

Related U.S. Application Data
(60) Provisional application No. 62/099,742, filed on Jan. 5, 2015.
(60) Provisional application No. 61/939,628, filed on Feb. 13, 2014.

(51) Int. Cl. C12M 1/42; C12Q 1/66

(52) U.S. Cl. CPC C12M 35/06; C12Q 1/66

(57) ABSTRACT

The present invention is directed generally to systems for making host cells with artificial endosymbionts. In an embodiment, the systems of the invention comprise a host cell, an artificial endosymbiont, a means for introducing the artificial endosymbiont to the host cell, a means for separating the host cells with artificial endosymbionts from free artificial endosymbionts, and a means for detecting artificial endosymbionts in host cells.

### FIELD OF THE INVENTION

The present invention relates generally to systems for making host cells with artificial endosymbionts. The present invention also relates to methods using the systems of the invention to make host cells with endosymbionts.

### BACKGROUND OF THE INVENTION

Mitochondria, chloroplast and other membrane bound organelles add heritable functionalities, such as photosynthesis, to eukaryotic cells. Such organelles (identified by their vestigial circular DNA) are believed to be endosymbiotically derived.

Bacteria exist with a wide range of functionalities not present in various eukaryotic cells. For example, in 1975 Blakemore identified magnetotactic bacteria (MTB) that orient and swim along a geomagnetic field. (Blakemore, R. Magnetotactic bacteria. Science 24: 377-379 (1975) (which is incorporated by reference in its entirety for all purposes)). These magnetotactic bacteria produce magnetic structures called magnetosomes that are composed of magnetite (Fe₃O₄) or greigite (Fe₃S₄) enclosed by a lipid membrane. A large number of MTB species have been identified since their initial discovery.

### SUMMARY OF THE INVENTION

The invention is related to systems for making host cells with artificial endosymbionts. In an embodiment, the systems of the invention comprises a host cell, an artificial endosymbiont, and a means for introducing the artificial endosymbiont to the host cell. In an embodiment, the systems of the invention comprise a host cell, an artificial endosymbiont, a means for introducing the artificial endosymbiont to the host cell, and a means for enriching host cells with artificial endosymbionts. In an embodiment, the system may further comprise a means for detecting host cells containing the artificial endosymbiont. In an embodiment, the systems of the invention comprise a host cell, an artificial endosymbiont, a means for introducing artificial endosymbionts to the host cell, and means for detecting host cells containing artificial endosymbionts. In an embodiment, the means for introducing the artificial endosymbiont can be a magnetic means, an acceleration means, an injection means, an electric field means, a ligand-receptor means, a concentration means, a chemical means, a membrane fusion means, and/or a phagosome escape means. In an embodiment, the means for detecting host cells containing artificial endosymbionts can be used to measure the efficiency, concentration, and/or quantity of artificial endosymbionts acquired by the host cell. In an embodiment, the means for detecting measures a thermal response from the host cells containing artificial endosymbionts after the application of a low frequency, alternating magnetic field. In an embodiment, the means for detecting utilizes MRI or NMR to measure the artificial endosymbionts in the host cells. In an embodiment, the means for detecting utilizes magnometry, hyperthermia, EPR, Mossbauer, SQUID or other magnetic measurement to measure the artificial endosymbionts in the host cells In an embodiment, the artificial endosymbiont expresses GFP or other optically active reporters and the means for detection makes optical measurements that measure the amount of light made by the GFP or other optically active reporter. In an embodiment, the artificial endosymbiont expresses a nonoptical reporter and the means for detecting measures the nonoptical reporter or a signal, molecule, or product from the nonoptical reporter.

In an embodiment, the means for introducing the artificial endosymbiont is a magnetic means arranged so that the artificial endosymbionts move towards the host cells. In an embodiment, the magnetic means may be a permanent magnet or an electromagnet. In an embodiment, the magnetic means is placed near or adjacent to a container which contains the host cells and artificial endosymbionts. In an embodiment, the magnetic means is designed to produce similar amounts of loading of artificial endosymbionts into host cells across the container. In an embodiment, the loading is similar in that portion of the container where artificial endosymbionts make contact with host cells. In an embodiment, the magnetic means is designed to provide a magnetic field strength that is similar in the container where the artificial endosymbionts are introduced into the host cells. In an embodiment, the magnetic means is designed and/or operated to provide a magnetic field strength that is similar across the container. In an embodiment, the magnetic means is designed and/or operated to provide a magnetic field gradient above the bottom or floor of the container that is similar across the container. In an embodiment, the magnetic field strength is uniform across the container. In an embodiment, the magnetic field distribution in each container is similar. In an embodiment, the magnetic field distribution is uniform in each container. In an embodiment, the system has a plurality of containers and the magnetic field distribution is similar in each container. In an embodiment, the system has a plurality of containers and the magnetic field distribution is uniform from container to container. In an embodiment, the loading of artificial endosymbionts into host cells is similar from container to container in the multiple container system. In an embodiment, the multiple containers are arranged in a multiwell plate format, which are well-known in the art, including formats with 2, 6, 12, 24, 48, etc. wells. In an embodiment, the plate is a six well plate in a 2 by 3 array. In an embodiment, the container is one suitable for a scale-up or an industrial application, such as, for example, a flask, fermenter or bioreactor.

The invention also relates to methods for making host cells with artificial endosymbionts using the systems of the invention. In an embodiment, the host cell and artificial endosymbiont are placed in the same container, subjected to a means for introducing the artificial endosymbiont to the host cells, and then host cells containing artificial endosymbionts are obtained. In an embodiment, the host cell and artificial endosymbiont are placed in the same container, subjected to a means for introducing the artificial endosymbiont to the host cells, and then host cells containing artificial endosymbionts are obtained from a means for enriching host cells with artificial endosymbionts. In an embodiment, the host cell and artificial endosymbiont are placed in the same container, subjected to a means for introducing the artificial endosymbiont to the host cells, and then host cells containing artificial endosymbionts are analyzed using a means for detecting host cells containing the artificial endosymbiont. In an embodiment, the host cells are adherent to the container surface(s). In an embodiment, the host cells grow in suspension and do not attach to the surfaces of the container. In an embodiment, the host cell population has both suspension and adherent cells and the host cells are found in suspension and attached to the surface(s) of the container. In an embodiment, the host cells are a heterogeneous population of adherent and suspension cells.

The invention is also related to kits for making host cells with artificial endosymbionts. Kits of the invention comprise consumable items of the systems for making host cells with artificial endosymbionts. Kits may also comprise items of the systems of the invention that need to be replaced from time to time. In an embodiment, the kits of the invention comprise 1, 2, 3, or more items for the systems of the invention.

### BRIEF DESCRIPTION OF THE DRAWINGS

FIG. 1A-C are schematic depictions of a system with a magnetic means for introducing artificial endosymbionts into host cells. FIG. 1A is an aerial view of the system, and FIG. 1B is an exploded view of the system. FIG. 1C depicts an interchangeable insert for holding the magnetic means which adapts the system for an alternative format plate.

FIG. 2 depicts a magnetic means of the invention.

### DETAILED DESCRIPTION OF THE INVENTION

The present invention is directed to systems for making host cells with an artificial endosymbiont.

### DEFINITIONS

As used herein, the term “artificial endosymbiont” refers to a single-celled organism that is or has been introduced into a host cell through human intervention, and which has been or can be transferred to daughter cells of the host cell through at least five cell divisions. In an embodiment, the artificial endosymbiont may be transferred through at least three or four cell divisions. In an embodiment, the artificial endosymbiont may be transferred through at least one or two cell divisions. Artificial endosymbionts give the host cell a desired phenotype, which phenotype is maintained in daughter cells of the host cell through at least one, two, three, four, or five cell divisions. The single-celled organism may secrete to and/or transport from the host cell polypeptide(s), nucleic acid(s), lipid(s), carbohydrate(s), amino acid(s), or other factor(s). This communication between the single-celled organism and the host cell can result in a desired phenotype for the host cell.

As used herein, the term “cellular life cycle” refers to series of events involving the growth, replication, and division of a eukaryotic cell. It is divided into five stages, known as G₀, in which the cell is quiescent, G₁ and G₂, in which the cell increases in size, S, in which the cell duplicates its DNA, and M, in which the cell undergoes mitosis and divides.

As used herein, the term “daughter cell” refers to cells that are formed by the division of a cell.

As used herein, the term “essential molecule” refers to a molecule needed by a cell for growth or survival.

As used herein, the term “fluorescent protein” refers to a protein that absorbs photons of one wavelength and emits photons of another wavelength.

As used herein, the term “genetically modified” refers to altering the DNA of a cell so that an observable or measurable property or characteristic of the cell is changed.

As used herein, the term “host cell” refers to an eukaryotic cell in which an artificial endosymbiont can reside.

As used herein the term “intracellular endosymbiont” refers to a single-celled organism that spends at least part of its natural life-cycle inside the cells of an eukaryotic organism.

As used herein, the term “intracellular pathogen” refers to bacteria that naturally infect a host organism resulting in a disease in the host organism, and during the infection some bacteria enter host cells where the bacteria grow and reproduce.

As used herein, the term “luciferase” refers to a protein that emits photons from a chemical event, e.g. chemo- or bio-luminescence.

As used herein, the term “magnet” or “magnetic means” means a composition or device that produces a magnetic field. The magnet or magnetic means may be a permanent magnet or an electromagnet.

As used herein, the term “magnetosome” refers to particles of magnetite (i.e., Fe₃ O₄) or greigite (Fe₃S₄) enclosed by a sheath or membrane, either as individual particles, in chains of particles, or in other arrangements of the particles.

As used herein, the term “magnetotactic bacteria” or “MTB” refers to the class of bacteria that use magnetic fields to orientate their swimming patterns.

As used herein, the term “magnetic bacteria” refers to bacteria that are able to respond to an external magnetic field.

As used herein, the term “mammal” refers to warm-blooded vertebrate animals all of which possess hair and suckle their young.

As used herein, the term “phenotype” refers to the set of observable characteristics of an individual resulting from the interaction of its genotype with the environment.

As used herein, the term “reporter” refers to a polypeptide or a molecule that confers on the host cell a phenotype that is easily measured. An “optical reporter” refers to a polypeptide or a molecule that can be measured optically.

As used herein, the term “secrete” refers to the passing of molecules or signals from one side of a membrane to the other side.

As used herein, the term “selective agent” refers to a molecule, a polypeptide, or a set of culture conditions that are lethal or inhibitory to artificial endosymbionts and/or host cells in the absence of a selectable marker.

As used herein, a “selectable marker” is a gene that when introduced into a cell confers a trait suitable for selection.

Unless specific definitions are provided, all other technical and scientific terms used herein have the same meaning as commonly understood by one of ordinary skill in the art.

The singular terms “a”, “an”, and “the” include plural referents unless context clearly indicates otherwise. Similarly, the word “or” is intended to include “and” (and vice versa) unless the context clearly indicates otherwise. Numerical limitations given with respect to concentrations or levels of a substance are intended to be approximate. Thus, where a concentration is indicated to be at least (for example) 10 μg, it is intended that the concentration be understood to be at least approximately or about 10 μg.

### Artificial Endosymbionts

Artificial endosymbionts of the invention include bacteria that are capable of surviving in a eukaryotic cell. In an embodiment, the artificial endosymbiont introduces a phenotype to the host cell. In an embodiment, this phenotype introduced by the artificial endosymbiont is maintained in daughter cells. In an embodiment, the host cell maintains the phenotype for at least 1, 2, 3, 4, 5, 6, 7, 8, 9, 10, 11, 12, 13, 14, 15, 16, 17, 18, 19, or 20 days. In an embodiment, the artificial endosymbiont can stably maintain the phenotype in host daughter cells through at least 1 cell division, or least 2 cell divisions, or least 3 cell divisions, or at least 4 division, or at least 5 divisions, or at least 6, 7, 8, 9, 10, 11, 12, 13, 14, 15, 16, 17, 18, 19, or 20 cell divisions. In another embodiment, the artificial endosymbiont can stably maintain phenotype in daughter cells through 3-5 divisions, or 5-10 divisions, or 10-15 divisions, or 15-20 divisions.

In an embodiment of the invention the artificial endosymbionts are Proteobacteria. In an embodiment of the invention the artificial endosymbiont is an α-Proteobacteria. In the current taxonomic scheme based on 16S rRNA, α-proteobacteria are recognized as a Class within the phylum Proteobacteria, and are subdivided into 7 main subgroups or orders (Caulobacterales, Rhizobiales, Rhodobacterales, Rhodospirillales, Rickettsiales, Sphingomonadales and Parvularculales). (Gupta, R. S. Phylogenomics and signature proteins for the alpha Proteobacteria and its main groups. BMC Microbiology, 7:106 (2007) (which is incorporated by reference in its entirety for all purposes)).

A large number of α-proteobacterial genomes that cover all of the main groups within α-proteobacteria have been sequenced, providing information that can be used to identify unique sets of genes or proteins that have distinctive characteristics of various higher taxonomic groups (e.g., families, orders, etc.) within α-proteobacteria. (Id.).

One example of α-proteobacteria is magnetotactic bacteria (MTB). MTB are a diverse group of bacteria that belong to different subgroups of the Proteobacteria and Nitrospirae phylum, and are mostly represented within the α-proteobacteria. MTB have a Gram-negative cell wall structure (inner membrane, periplasm, and outer membrane). They inhabit water bodies or sediments with vertical chemical concentration gradients, are found predominantly at the oxic-anoxic interface and thus are categorized as microaerophiles, anaerobic, or facultative aerobic or some combination of the three. All MTB are chemoorganoheterotrophic although some marine strains can also grow chemolithoautotrophically (Bazylinski et al., 2004; Williams et al., 2006). All MTB contain magnetosomes, which are intracellular structures comprising magnetic iron crystals enveloped by a phospholipid bilayer membrane (Gorby et al., 1988).

A large number of MTB species are known to those of ordinary skill in the art since their initial discovery in 1975 by Blakemore (Blakemore, R. Magnetotactic bacteria. Science 24: 377-379 (1975) (which is incorporated by reference in its entirety for all purposes)) and represent a group of microbes (Faivre, D. & Schüler, D. Magnetotactic bacteria and magnetosomes. Chemistry Reviews 108: 4875-4898 (2008) (which is incorporated by reference in its entirety for all purposes)). MTB have been identified in different subgroups of the Proteobacteria and the Nitrospira phylum with most of the phylotypes grouping in α-Proteobacteria. Currently, culturable MTB strains assigned as α-Proteobacteria by 16S rRNA sequence similarity include the strain originally isolated by Blakemore in 1975, Magnetospirillum magnetotactium (formerly Aquasprillium magnetotactium), M. gryphiswaldense, M. magneticum strain AMB-1, M. polymorphum, Magnetosprillum sp. MSM-4 and MSM-6, Magnetococcus marinus, marine vibrio strains MV-1 and MV-2, a marine spirillum strain MMS-1 and Magnetococcus sp. strain MC-1, as well as others.

Other single-celled organisms useful as artificial endosymbionts include, for example, Anabaena, Nostoc, Diazotroph, Cyanobacteria, Trichodesmium, Beijerinckia, Clostridium, Green sulfur bacteria, Azotobacteraceae, Rhizobia, Frankia, flavobacteria, Methanosarcinales, aerobic halophilic Archaea of the order Halobacteriales, the fermentative anerobyves of the order Halanaerobiales (low G+C brand of the Firmicutes), the red aerobic Salinibacter (Bacteroidetes branch), Marinobacter, Halomonas, Dermacoccus, Kocuria, Micromonospora, Streptomyces, Williamsia, Tskamurella, Alteromonas, Colwellia, Glaciecola, Pseudoalteromonas, Shewanella, Polaribacter, Pseudomonas, Psychrobacter, Athrobacter, Frigoribacterium, Subtercola, Microbacterium, Rhodoccu, Bacillus, Bacteroides, Propionibacterium, Fusobacterium, Klebsiella, lecithinase-positive Clostridia, Veillonella, Fusobacteria, Chromatiaceae, Chlorobiceae, Rhodospirillaceae, thiobacilli, nitrosomonas, nitrobacter, Synechococcus (e.g., Synechococcus elongates), methanogens, acetogens, sulfate reducers, and lactic acid bacteria.

The genomes of a number of these single-celled organisms have been or are being sequenced, including for example: M. frigidum, M. burtonii, C. symbiosum, C. psychrerythraea, P. haloplanktis, Halorubrum lacusprofundi, Vibrio salmonicida, Photobacterium profundum, S. violacea, S. frigidimarina, Psychrobacter sp. 273-4, S. benthica, Psychromonas sp. CNPT3, Moritella sp., Desulfotalea Psychrophila, Exiguobacterium 255-15, Flavobacterium psychrophilum, Psychroflexus torquis, Polaribacter filamentous, P. irgensii, Renibacterium salmoninarum, Leifsonia-related PHSC20-cl, Acidithiobacillus ferrooxidans, Thermoplasma acidophilum, Picrophilus torridus, Sulfolobus tokodaii, and Ferroplasma acidarmanus.

Many intracellular pathogens include in their genome genomic islands containing virulence genes encoding, for example, adherence factors that allow the intracellular pathogen to attach to target eukaryotic cells, and trigger phagocytosis of the intracellular pathogen. (Juhas, M., van der Meer, J. R., Gaillard, M., Harding, R. M., Hood, D. W., Crook, D. W., Genomic islands: tools of bacterial horizontal gene transfer and evolution, FEMS Microbiol. Rev. 33:376-393 (2009), which is incorporated by reference in its entirety for all purposes.) Many virulence factors utilize type III or type IV secretion systems. Some virulence factors are secreted into the eukaryotic host cell and alter membrane traffic within the target eukaryotic cell, some virulence factors interact with host proteins involved in apoptosis. (Dubreuil, R. and Segev, N., Bringing host-cell takeover by pathogenic bacteria to center stage, Cell. Logis. 1:120-124 (2011), which is incorporated by reference in its entirety for all purposes.)

In an embodiment, artificial endosymbionts exclude single-celled organisms that are known to be intracellular pathogens or intracellular endosymbionts.

### Modified Artificial Endosymbionts

In an embodiment, α-proteobacteria are modified to enhance their function as artificial endosymbionts. Methods for genetically modifying artificial endosymbionts are well-known in the art. In an embodiment, modifications may involve increasing or decreasing production of proteins or RNA through changing promoter or ribosome binding sequences, or deleting or silencing certain genes in the artificial endosymbiont. In an embodiment, modifications involve mutations that change a polypeptide in the artificial endosymbiont resulting in a desired trait for the artificial endosymbiont. For example, a desired trait of the artificial endosymbiont could be nutrient, factor, cofactor or other molecule dependence on the host cell. In an embodiment, the desired trait allows the artificial endosymbiont to make a nutrient, factor, cofactor or other molecule needed by the host cell. In some embodiments, the desired trait creates a phenotype in the host cell, while in other embodiments the desired trait does not create a phenotype in the host cell.

In one embodiment, the flagellar proteins of an artificial endosymbiont are modified so that the flagellar proteins are no longer expressed. In an embodiment, the single-celled organism is modified so that it can no longer synthesize an essential molecule that is preferably provided by the eukaryotic host cell. In an embodiment, the single-celled organism is genetically modified so that its cell cycle is coordinated with the cell cycle of the eukaryotic host cell so that copy number of the single-celled organism can be maintained at a sufficient level to impart the phenotype to daughter cells.

Molecular biology tools have been developed for genetic manipulations of MTB most extensively in AMB-1 and M. gryphiswaldense strain MSR-1 (reviewed in Jogler, C. and Schtiler, D. in Magnetoreception and Magnetosomes in Bacteria, New York, Springer, 2007 p 134-138 (which is incorporated by reference in its entirety for all purposes)). The genomes of two other Magnetospirillum strains and Magnetococcus sp. strain MC-1 have also been recently sequenced. In an embodiment, genes from these strains or other MTB strains, presently culturable or unculturable, sequenced or unsequenced, know or unknown, can be used in the present invention.

In an embodiment, the artificial endosymbionts of the invention are modified with a cholesterol-dependent cytolysin (CDC) found in a number of Gram-positive pathogens such as, for example, Listeria monocytogenes (listeriolysin O or LLO). In an embodiment, LLO is recombinantly produced. In an embodiment, LLO is added to the artificial endosymbiont so that the LLO coats the artificial endosymbiont. In an embodiment, artificial endosymbionts are mixed with 0.1, 0.2, 0.3, 0.4, 0.5, 0.6, 0.7, 0.8, 0.9, 1.0, 2.0, 3.0, 4.0, or 5.0 μM LLO. The LLO may be at concentration ranges of 0.1-5.0 μM, or 0.1-1.0 μM, 1.0-5.0 μM, or 1.0-2.0 μM. In an embodiment, the artificial endosymbiont is an MTB, and the MTB is coated with LLO. In an embodiment, the artificial endosymbiont is coated with an appropriate serum albumin, such as, for example, bovine serum albumin, or other appropriate carrier protein. Following coating with the serum albumin or other carrier protein, the LLO is coated onto the artificial endosymbiont. In an embodiment, the serum albumin or other carrier protein is from the same species as the host cell.

In an embodiment, the artificial endosymbiont is modified to recombinantly express LLO. In an embodiment, the artificial endosymbiont is modified to recombinantly express and secrete LLO. In an embodiment, an MTB is modified to recombinantly express LLO. In an embodiment, and MTB is modified to recombinantly express and secrete LLO.

In an embodiment, the artificial endosymbiont is mixed with an agent that raises the pH of lysosomes in the host cell. In an embodiment, the agent is, for example, Bafilomycin, monensin, nigericin, amantadine, chloroquine, methylamine, or ammonium chloride. In an embodiment, the agent is used at a concentration of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 μM. The agent may be used in a range of 0.1-10.0 μM, or 0.1-1.0 μM, or 1.0-10.0 μM, or 1.0-5.0 μM. In an embodiment, the methylamine, ammonium chloride or other such agent is used at a concentration of 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, or 10.0 mM. Such agents may be used in concentration ranges of 0.1-10 mM, 0.1-1.0 mM, or 1.0-10.0 mM, or 1.0-5.0 mM.

### Nucleic Acids

The nucleic acids of the invention can include expression vectors, such as plasmids, or viral vectors, or linear vectors, or vectors that integrate into chromosomal DNA. Expression vectors can contain a nucleic acid sequence that enables the vector to replicate in one or more selected host cells. Such sequences are well-known for a variety of cells. The origin of replication from the plasmid pBBR1MCS and pBBR1MCS-2 (Kovach et al., 1995, Gene 166:175-176, which is hereby incorporated by reference in its entirety for all purposes) are suitable for most Gram-negative bacteria. In eukaryotic host cells, e.g., mammalian cells, the expression vector may integrate into the host cell chromosome where the expression vector replicates with the host chromosome. Similarly, expression vectors can be integrated into the chromosome of prokaryotic cells where the vector replicates with the chromosome.

Expression vectors also generally contain a selection gene, also termed a selectable marker. Selectable markers are well-known in the art for prokaryotic and eukaryotic cells, including host cells of the invention. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous gene produce a protein conferring drug resistance and thus survive the selection regimen. Other selectable markers for use in bacterial or eukaryotic (including mammalian) systems are well-known in the art. In some embodiments, the selectable marker is a protein that is secreted by the artificial endosymbiont or encoded by a nucleic acid that is secreted by the artificial endosymbiont into the host cell.

The expression vector for producing a heterologous polypeptide may also contain an inducible promoter that is recognized by the host RNA polymerase and is operably linked to the nucleic acid encoding the target protein. Inducible or constitutive promoters (or control regions) with suitable enhancers, introns, and other regulatory sequences are well-known in the art.

In some embodiments, it may be desirable to modify the polypeptides. One of skill will recognize many ways of generating alterations in a given nucleic acid construct. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Giliman and Smith, 1979, Gene 8:81-97, Roberts et al., 1987, Nature 328: 731-734 (both of which are incorporated by reference in their entirety for all purposes).

In some embodiments, the recombinant nucleic acids encoding the polypeptides of the invention are modified to provide preferred codons for a particular cell, which enhance translation of the nucleic acid in the selected cell.

The polynucleotides of the invention also include polynucleotides including nucleotide sequences that are substantially equivalent to the polynucleotides of the invention. Polynucleotides according to the invention can have at least about 80%, more typically at least about 90%, and even more typically at least about 95%, sequence identity to a polynucleotide of the invention. The invention also provides the complement of the polynucleotides including a nucleotide sequence that has at least about 80%, more typically at least about 90%, and even more typically at least about 95%, sequence identity to a polynucleotide encoding a polypeptide recited above. The polynucleotide can be DNA (genomic, cDNA, amplified, or synthetic) or RNA. Methods and algorithms for obtaining such polynucleotides are well-known to those of skill in the art and can include, for example, methods for determining hybridization conditions which can routinely isolate polynucleotides of the desired sequence identities.

Nucleic acids which encode protein analogs in accordance with this invention (i.e., wherein one or more amino acids are designed to differ from the wild type polypeptide) may be produced using site directed mutagenesis or PCR amplification in which the primer(s) have the desired point mutations. For a detailed description of suitable mutagenesis techniques, see Sambrook et al., 1989, Molecular Cloning: A Laboratory Manual, Cold Spring Harbor Laboratory Press, Cold Spring Harbor, N.Y. and/or Ausubel et al., editors, 1994, Current Protocols in Molecular Biology, Green Publishers Inc. and Wiley and Sons, N.Y. Chemical synthesis using methods described by Engels et al., 1989, in Angew. Chem. Intl. Ed., Volume 28, pages 716-734 (all of which are incorporated by reference in their entirety for all purposes), may also be used to prepare such nucleic acids.

“Recombinant variant” refers to any polypeptide differing from naturally occurring polypeptides by amino acid insertions, deletions, and substitutions, created using recombinant DNA techniques. Guidance in determining which amino acid residues may be replaced, added, or deleted without abolishing activities of interest, such as enzymatic or binding activities, may be found by comparing the sequence of the particular polypeptide with that of homologous peptides and minimizing the number of amino acid sequence changes made in regions of high homology.

Preferably, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

“Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides or chimeric polypeptides. Such alterations can, for example, alter one or more of the biological functions or biochemical characteristics of the polypeptides of the invention. For example, such alterations may change polypeptide characteristics such as ligand-binding affinities or degradation/turnover rate. Further, such alterations can be selected so as to generate polypeptides that are better suited for expression, scale up and the like in the host cells chosen for expression.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, or degradation/turnover rate.

In a preferred method, polynucleotides encoding the novel nucleic acids are changed via site-directed mutagenesis. This method uses oligonucleotide sequences that encode the polynucleotide sequence of the desired amino acid variant, as well as sufficient adjacent nucleotide(s) on both sides of the changed amino acid to form a stable duplex on either side of the nucleotide(s) being changed. In general, the techniques of site-directed mutagenesis are well-known to those of skill in the art and this technique is exemplified by publications such as, Edelman et al., 1983, DNA 2:183 (which is incorporated by reference in its entirety for all purposes). A versatile and efficient method for producing site-specific changes in a polynucleotide sequence was published by Zoller and Smith, 1982, Nucleic Acids Res. 10:6487-6500 (which is incorporated by reference in its entirety for all purposes).

PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the target at the position specified by the primer. The product DNA fragments replace the corresponding region in the plasmid and this gives the desired amino acid variant.

A further technique for generating amino acid variants is the cassette mutagenesis technique described in Wells et al., 1985, Gene 34:315 (which is incorporated by reference in its entirety for all purposes); and other mutagenesis techniques well-known in the art, such as, for example, the techniques in Sambrook et al., supra, and Current Protocols in Molecular Biology, Ausubel et al. (which is incorporated by reference in its entirety for all purposes).

### Host Cells

The invention provides a eukaryotic host cell containing an artificial endosymbiont wherein the artificial endosymbiont imparts a phenotype to the host cell. In an embodiment, the artificial endosymbiont is heritable.

In some embodiments the host cells of the invention are animal cells. In some embodiments the host cells are mammalian, such as mouse, rat, rabbit, hamster, human, porcine, bovine, or canine. Mice routinely function as a model for other mammals, most particularly for humans. (See, e.g., Hanna, J., Wernig, M., Markoulaki, S., Sun, C., Meissner, A., Cassady, J. P., Beard, C., Brambrink, T., Wu, L., Townes, T. M., Jaenisch, R. Treatment of sickle cell anemia mouse model with iPS cells generated from autologous skin. Science 318: 1920-1923 (2007); Holtzman, D. M., Bales, K. R., Wu, S., Bhat, P., Parsadanian, M., Fagan, A., Chang, L. K., Sun, Y., Paul, S. M. Expression of human apolipoprotein E reduces amyloid-β deposition in a mouse model of Alzheimer's disease. J. Clin. Invest. 103(6): R15-R21 (1999); Warren, R. S., Yuan, H., Matli, M. R., Gillett, N. A., Ferrara, N. Regulation by vascular endothelial growth factor of human colon cancer tumorigenesis in a mouse model of experimental liver metastasis. J. Clin. Invest. 95: 1789-1797 (1995) (each of these publications is incorporated by reference in its entirety for all purposes)). Animal cells include, for example, fibroblasts, epithelial cells (renal, mammary, prostate, lung), keratinocytes, hepatocytes, adopicytes, endothelial cells, and hematopoietic cells.

In some embodiments, the host cell is a cancer cell, including human cancer cells. There are many cancer cell lines that are well-known to those of ordinary skill in the art, including common epithelial tumor cell lines such as Coco-2, MDA-MB231 and MCF7, non-epithelial tumor cell lines, such as HT-1080 and HL60, and the NCI60-cell line panel (see, e.g., Shoemaker, R., The NCI60 human tumor cell line anticancer drug screen. Nature Reviews Cancer 6, 813-823 (2006) (which is incorporated by reference in its entirety for all purposes)). Additionally, those of ordinary skill in the art are familiar with obtaining cancer cells from primary tumors. Cancer cells also include, for example, solid tumor cell types, hematopoietic cancer cells, carcinomas, sarcomas, leukemias, lymphomas, gliomas, as well as specific tissue related cancers such as prostate cancer, breast cancer, lung cancer, colorectal cancer, pancreatic cancer, melanoma, glyoblastoma, liver cancer, and the types of cancer cells in the NCI 60 panel of cancer cell lines.

In other embodiments, the host cells are stem cells. Those of ordinary skill in the art are familiar with a variety of stem cell types, including for example, embryonic stem cells, inducible pluripotent stem cells, hematopoietic stem cells, neural stem cells, epidermal neural crest stem cells, mammary stem cells, intestinal stem cells, mesenchymal stem cells, olfactory adult stem cells, testicular cells, and progenitor cells (e.g., neural, angioblast, osteoblast, chondroblast, pancreatic, epidermal).

In an embodiment, the host cell is a cell found in the circulatory system of a mammal, including humans. For example, red blood cells, platelets, plasma cells, T-cells, natural killer cells, or the like, and precursor cells of the same. As a group, these cells are defined to be circulating host cells of the invention. The present invention may be used with any of these circulating cells. In an embodiment, the host cell is a T-cell. In another embodiment, the host cell is a B-cell. In an embodiment the host cell is a neutrophil. In an embodiment, the host cell is a megakaryocyte.

In an embodiment the host cell is a plant cell. In some embodiments the host cells are cells of monocotyledonous or dicotyledonous plants including, but not limited to, maize, wheat, barley, rye, oat, rice, soybean, peanut, pea, lentil and alfalfa, cotton, rapeseed, canola, pepper, sunflower, potato, tobacco, tomato, eggplant, eucalyptus, a tree, an ornamental plant, a perennial grass, or a forage crop. In other embodiments the host cells are algal, including but not limited to algae of the genera Chlorella, Chlamydomonas, Scenedesmus, Isochrysis, Dunaliella, Tetraselmis, Nannochloropsis, or Prototheca, In some embodiments the host cells are fungi cells, including but not limited to fungi of the genera Saccharomyces, Klyuveromyces, Candida, Pichia, Debaromyces, Hansenula, Yarrowia, Zygosaccharomyces, or Schizosaccharomyces.

In another embodiment, at least one gene from the host cell is genetically altered. In some embodiments, mutual nutritional dependence (biotrophy) may be established between the artificial endosymbiont and the host cell by genetic modification of the host cell, using the appropriate molecular biology techniques specific to the target host cell type known to those of ordinary skill in the art, creating host cell dependence on the artificial endosymbiont for some essential macromolecule thus establishing the environmental pressures for biotrophy. In another embodiment, nutritional dependence for an artificial endosymbiont on the host cell may be established by genetically altering the host cell to eliminate the ability of the host cell to synthesize various metabolites, cofactors, vitamins, nucleotides, or other essential molecules. In such embodiments, the essential molecule may be provided by the artificial endosymbiont. In another embodiment, the host cell gene encoding the enzyme serine hydroxymethyltransferase, which converts serine into glycine at the terminus of the 3-phosphoglycerate biosynthetic pathway for amino acid production, may be modified.

Systems for Making Host Cells with Artificial Endosymbionts

The systems of the invention allow artificial endosymbionts to be introduced into host cells. In an embodiment, the systems of the invention comprise an artificial endosymbiont, a host cell, and a means for introducing the artificial endosymbiont into the host cell. The systems of the invention allow artificial endosymbionts to be introduced into host cells. In an embodiment, the systems of the invention comprise an artificial endosymbiont, a host cell, a means for introducing the artificial endosymbiont into the host cell, and a means for enriching the host cells with the endosymbiont. In an embodiment, the systems of the invention comprise an artificial endosymbiont, a host cell, a means for introducing the artificial endosymbiont into the host cell, and a means for detecting host cells containing the artificial endosymbiont.

The means for introducing the artificial endosymbiont includes means for assisting in introduction of the artificial endosymbionts into the host cells. Examples of means for introducing include magnetic means for assisting introduction, acceleration means for assisting introduction, electric field means for assisting introduction, injection means for assisting introduction, ligand-receptor means for assisting introduction, concentration means for assisting introduction, chemical means for introduction, phagosome escape means for introduction, and membrane fusion means for assisting introduction. Some means for introducing may fall in more than one of these categories.

In an embodiment, the magnetic means of the invention is a magnet. In an embodiment, the magnet may be a permanent magnet or an electromagnet. In an embodiment, a magnet or a plurality of magnets are adapted for use with a container. In an embodiment, the container may be in a microtiter dish, petri dish, cell culture dish, cell culture flask or other apparatus known to those of ordinary skill in the art. In an embodiment, the microtiter dish has 6, 24, 48, 96, 384, 1536, 3456, or 9600 wells (or containers). In an embodiment, the microtiter dish includes any commercially available format. In an embodiment, the magnetic means is designed to fit one microtiter plate, or it may be designed to fit a plurality of microtiter plates. In this embodiment, the magnetic means may be a single magnet or may be a plurality of magnets sized for use with individual wells, or sized for an individual plate or a plurality of plates, or sized for some amount of wells in a plate. In an embodiment, the magnetic means is adapted for use with a test tube or other container. In this embodiment, test tubes include any commercially available tubes including microcentrifuge tubes, glass, Pyrex, plastic or other test tubes. In this embodiment, the magnetic means may be adapted for use with a single tube or a plurality of tubes. When a plurality of tubes is used, the tubes may be held in a rack, and the magnetic means will be designed to be compatible with the rack. In an embodiment, the container holds a volume of liquid suitable for the application. In an embodiment, the container holds at least 0.1 ml, at least 0.5 ml, at least 1.0 ml, at least 2.0 ml, at least 5.0 ml, at least 10.0 ml, at least 25.0 ml, at least 40 ml, at least 50.0 ml, at least 100 ml, or at least 1000 ml, or 2, 3, 4, 5, 6, 7, 8, 9, or 10 liters, or 20, 30, 40, 50, 60, 70, 80, 90 or 100 liters, or 200, 300, 400, 500, 600, 700, 800, 900, or 1000 liters, or 2,000, 3,000, 4,000, 5,000, 6,000, 7,000, 8,000, 9,000 or 10,000 liters, or 20,000, 30,000, 40,000 or 50,000 liters. In this embodiment, the magnetic means will be designed to be compatible with the container. In this embodiment, the magnetic means is one or a plurality of magnets.

In an embodiment, the magnetic means creates a high magnetic field gradient in the containers. In an embodiment, the magnetic means provides a similar magnetic field strength across an individual container (e.g., a single well of a microtiter dish) and a high gradient moving in the vertical direction over the container. In an embodiment, the magnetic means provides a similar magnetic field strength and gradient across each container in the system, e.g., across each well of a microtiter dish or multiwell culture plate or each tube in the system. In an embodiment, the magnetic means provides a uniform magnetic field strength across each plate in the system.

In an embodiment, the magnetic means has the following properties: (1) a large field strength and gradient, (2) a similar field strength and gradient across each individual container in the system, and (3) a similar field strength and gradient across each container of the system. In an embodiment, the magnetic means is an array of magnetic means. In an embodiment, the periodicity of the magnetic means array matches the periodicity of the wells in the container array. In an embodiment, the magnetic means is a NdFeB permanent magnet or an electromagnet that extends slightly beyond the edges of the container. In an embodiment, the NdFeB permanent magnet has layers where at least some of the layers have a reduced area compared to other layers. In an embodiment, the top layer of the NdFeB permanent magnet is the largest and each subsequence layer has a reduced area compared to the layer above it. In an embodiment, each layer of the NdFeB permanent magnet has the same thickness as the other layers. In an embodiment, at least some of the layers of the NdFeB permanent magnet have different thicknesses.

In an embodiment, the magnetic means produces a magnetic field strength of 0.01-1 Tesla. In an embodiment, the magnetic means produces an oscillating magnetic field of 0-100 Hz, in an embodiment, the magnetic means produces a magnetic field having a low frequency (about 2 Hz), and small amplitude (about 0.5-3 mm) of magnet oscillation. In an embodiment, the magnetic means produces an oscillating magnetic field, for example, using a NanoTherics Ltd. products for producing oscillating magnetic fields. The oscillating magnetic field can also be created, for example, by varying the current in an electromagnet or by mechanically vibrating a permanent magnet.

In an embodiment, the density of magnetic bacteria is increased such that the movement of magnetic bacteria towards the eukaryotic host cells is increased. In this embodiment, the magnetic bacteria interact collectively with the magnetic field producing higher flow velocities. If the magnetic bacteria are tightly packed the group will see a magnetic force many times that experienced by a single magnetic bacteria.

In an embodiment, the system includes magnetic shield materials to protect the user and other equipment/devices from the magnetic field of the magnetic means. Such magnetic shield materials include, for example, Giron Magnetic Shielding film, MuMetal®, Co-Netic®, Netic®, MagnetShield, PaperShield, and other materials well-known in the art and/or commercially available. In an embodiment, the magnetic shield materials are arranged so that equipment/devices next to the systems of the invention are protected from the magnetic field of the magnetic means in the system. In an embodiment, the NdFeB permanent magnet is cylindrical. In an embodiment, the layers of the NdFeB permanent magnet are cylindrical.

In an embodiment, the acceleration means for assisting introduction is a centrifuge or other means for accelerating the artificial endosymbiont. In an embodiment, a magnet or magnetic means can be used to accelerate the artificial endosymbiont. In this embodiment, the artificial endosymbiont can be a magnetic bacteria. In an embodiment, electroporation or magnetic fields are used to accelerate the artificial endosymbiont. (Potrykus, I. Gene transfer methods for plants and cell cultures. Ciba Found Symp 154, 198-208; discussion 208-112 (1990); Wolbank, S. et al. Labeling of human adipose-derived stem cells for non-invasive in vivo cell tracking. Cell Tissue Bank 8, 163-177 (2007) (each of these two publications is incorporated by reference in its entirety for all purposes)). In an embodiment, intracellular delivery can be achieved by bombarding cells or tissues with accelerated molecules or bacteria without the need for carrier particles. (Lian, W. N., Chang, C. H., Chen, Y.-J., Dao, R.-O., Luo Y.-C., Chien, J.-Y., Hsieh, S.-L., Lin, C.-H., Intracellular delivery can be achieved by bombarding cells or tissues with accelerated molecules or bacteria without the need for carrier particles, Experimental Cell Research 313(1): 53-64 (2007); Heng, B. C. & Cao, T. Immunoliposome-mediated delivery of neomycin phosphotransferase for the lineage-specific selection of differentiated/committed stem cell progenies: Potential advantages over transfection with marker genes, fluorescence-activated and magnetic affinity cell-sorting. Med. Hypotheses 65(2): 334-336 (2005); Potrykus (1990) Ciba Found Symp, Vol. 1 54: 198 (each of these two publications is incorporated by reference in its entirety for all purposes)).

In an embodiment, electroporation is used as an electric field means to assist in introduction of the artificial endosymbiont. (Potrykus, I. Gene transfer methods for plants and cell cultures. Ciba Found Symp 154, 198-208; discussion 208-112 (1990); Wolbank, S. et al. Labeling of human adipose-derived stem cells for non-invasive in vivo cell tracking. Cell Tissue Bank 8, 163-177 (2007) (each of these two publications is incorporated by reference in its entirety for all purposes)).

In an embodiment, an injection means is used to assist in the introduction of an artificial endosymbiont into a host cell. In an embodiment, the injection means is a microinjector. A variety of microinjectors, microinjector systems, and microinjection techniques are well-known to those skilled in the art. Microinjection is the most efficient transfer technique available (essentially 100%) (Id.; Xi, Z. & Dobson, S. Characterization of Wolbachia transfection efficiency by using microinjection of embryonic cytoplasm and embryo homogenate. Appl. Environ. Microbiol. 71(6): 3199-3204 (2005); Goetz, M., Bubert, A., Wang, G., Chico-Calero, I., Vazquez-Boland, J. A., Beck, M., Slaghuis, J., Szalay, A. A., Goebel, W. Microinjection and growth of bacteria in the cytosol of mammalian host cells. Proc. Natl. Acad. Sci. USA 98:12221-12226 (2001); Microinjection and Organelle Transplantation Techniques: methods and applications, Eds. J. E. Celis, A. Graessmann, and A. Loyter, Academic Press (1986) (each of these publications is incorporated by reference in its entirety for all purposes)).

In an embodiment, a ligand-receptor means is used to assist in introduction of an artificial endosymbiont into a host cell. Some endosymbionts, pathogens, or parasites have their own methods for cellular entry and these natural processes can be exploited for internalization of the artificial endosymbionts resulting in the generation of so-called symbiosomes. These natural processes can involve host cell receptors and ligands that the bacteria express on their cell surface. In an embodiment, artificial endosymbionts are modified to recombinantly express the ligand on the cell surface, and the modified artificial endosymbiont enters the host cell through this pathway. Examples of such endosymbiont or parasite ligands include InlA and InlB from Listeria monocytogenes and Pla from Yersinia pestis. (Mengaud J, Ohayon H, Gounon P, Mege R M, Cossart P. E-cadherin is the receptor for internalin, a surface protein required for entry of L. monocytogenes into epithelial cells. Cell 1996; 84:923-32. Braun L, Ghebrehiwet B, Cossart P. gC1q-R/p32, a C1q-binding protein, is a receptor for the InlB invasion protein of Listeria monocytogenes. EMBO J 2000; 19:1458-66. Shen Y, Naujokas M, Park M, Ireton K. InIB-dependent internalization of Listeria is mediated by the Met receptor tyrosine kinase. Cell 2000; 103:501-10. Lahteenmaki K, Kukkonen M, Korhonen T K. The Pla surface protease/adhesin of Yersinia pestis mediates bacterial invasion into human endothelial cells. FEBS letters 2001; 504:69-72. Cowan C, Jones H A, Kaya Y H, Perry R D, Staley S C. Invasion of epithelial cells by Yersinia pestis: evidence for a Y. pestis-specific invasin. Infection and Immunity 2000; 68:4523-30; Advances in Molecular and Cellular Microbiology, Ed. Richard J. Lamont, 2004, Intracellular Niches of Microbes: A Pathogens Guide Through the Host Cell, Eds. Ulrich Schaible and Albert Haas, 2009, Wiley-VCH, Weinheim, pg. 712 (each of these publications is incorporated by reference in its entirety for all purposes)). Other invasion proteins and genes that are well known in the art can also be used in the invention to facilitate introduction of an artificial endosymbiont into a host cell. For example, Listeriolysin O (LLO) can be engineered into the artificial endosymbiont to facilitate release of the artificial endosymbiont from the host cell phagosome. In an embodiment, the Listeriolysin O (LLO) originates from Listeria monocytogenes, or from other pathogens well known in the art to have similar lysin proteins.

Intracellular bacteria have evolved mechanisms to survive and replicate in host cells. Intracellular bacteria can enter the host cell via phagocytosis and the nascent phagosome is directed into the endocytic pathway. Phagosomes bearing nonpathogens will follow a path that fuses with the lysosomal compartment resulting in bacterial degradation. Pathogens have developed different strategies to avoid this fate. A few pathogenic bacteria including Shigella, Listeria, and Francisella, escape from the phagosome into the cytosol of the host cell. Although these pathogens avoid the endocytic pathway and the challenge of residing in a nutrient poor vacuolar compartment. Most of the characterized intracellular pathogens remain within a membrane-bound compartment and modify this niche to facilitate their survival and replication. These factors can be expressed in the artificial endosymbiont to aid in survival of artificial endosymbiont.

Escape from the phagosome is a bacterially driven process, and pathogens share common mechanisms for escape that are triggered by specific environmental cues inside the phagosome. Cytosolic pathogens rely on the production of secreted enzymes to escape the phagosome and reach the cytosol. These includes secreted enzymes include listeriolysin O (LLO), phosphatidylinositol specific phospholipase C (PI-PLC), phosphatidylcholine-specific phospholipase C (PC-PLC) by Listeria monocytogenes, secretion of type 3 secretion system effector IpaB (hemolysin) by Shigella flexneri, secretion of protein of unknown function BPSS1539 by Burkholderia pseudomallei, secretion of hemolysin C and phospholipases by Rickettsia prowazekii, IglC, MglA and FTT1103 by Francisella tularensis.

Other bacterial factors secreted by bacteria for host cell invasion are invasins (mostly enzymes). Invasins facilitate the replication and spread of bacteria and act locally in the immediate vicinity of bacteria. Examples of invasins include hyaluronidase (Streptococci, Staphylocci), collagenase (Clostridium), neuraminidase (Vibrio and Shigella), coagulase (Staphlylococcus), leukocidin (Staphylococcus), streptolysin (Streptococcus), hemolysins (Clostridia), lecithinases (Clostridia), phospholipases (Clostridia), anthrax EF (Bacillus anthracis), perussis ASC (Bordetella pertussis), and internalins (InlA, InlB of L. monocytogenes).

In an embodiment, the modified artificial endosymbiont expresses a ligand on its surface that interacts with a host cell receptor allowing the modified artificial endosymbiont to be internalized. Examples of such ligands are InlA and InlB from Listeria monocytogenes and Pla from Yersinia pestis.

In an embodiment, the concentration means produces an increase in the local concentration of the artificial endosymbiont near the host cells. In an embodiment the concentration means also increases the local concentration of host cells. Examples of means for increasing the local concentration include filters, membranes, vacuum pumps, other drying devices, columns, centrifuges and magnets. In an embodiment, the volume containing the host cells and artificial endosymbionts is reduced as fluid passes through a filter or membrane, which filter or membrane retains the host cells and artificial endosymbionts but allows fluid to pass. In an embodiment, the volume containing the host cells and artificial endosymbionts is reduced by evaporation using a vacuum apparatus and/or drying apparatus.

In an embodiment, a membrane fusion means is used to assist in the introduction of the artificial endosymbiont into the host cell. In an embodiment, artificial endosymbionts are introduced to a host cell by a liposome mediated process. Mitochondria and chloroplasts have been efficiently introduced into eukaryotic cells when encapsulated into liposomes. (Bonnett, H. T. Planta 131, 229 (1976); Giles, K.; Vaughan, V.; Ranch, J.; Emery, J. Liposome-mediated uptake of chloroplasts by plant protoplasts. In Vitro Cellular & Developmental Biology—Plant 16(7) 581-584 (each of these two publications is incorporated by reference in its entirety for all purposes)). Numerous liposome fusion protocols and agents are available and can be used by the skilled artisan without undue experimentation. (See, e.g., Ben-Haim, N., Broz, P., Marsch, S., Meier, W., Hunziker, P. Cell-specific integration of artificial organelles based on functionalized polymer vesicles. Nano Lett. 8(5): 1368-1373 (2008), which is incorporated by reference in its entirety for all purposes). Intracellular delivery can be achieved by bombarding cells or tissues with accelerated molecules or bacteria without the need for carrier particles. (Lian, W., Chang, C., Chen, Y., Dao, R., Luo, Y., Chien, J., Hsieh, S., Lin, C. Experimental Cell Research 313(1): 53-64 (2007); Heng, B. C. & Cao, T. Immunoliposome-mediated delivery of neomycin phosphotransferase for the lineage-specific selection of differentiated/committed stem cell progenies: Potential advantages over transfection with marker genes, fluorescence-activated and magnetic affinity cell-sorting. Med. Hypotheses 65(2): 334-336 (2005); Potrykus (1990) Ciba Found Symp, Vol. 1 54: 198 (each of these publications is incorporated by reference in its entirety for all purposes)). Additionally, liposome uptake can be enhanced by manipulation of incubation conditions, variation of liposome charge, receptor mediation, and magnetic enhancement. (See, e.g., Pan et al. Int. J. Pharm. 358: 263 (2008); Sarbolouki, M. N. & Toliat, T. Storage stability of stabilized MLV and REV liposomes containing sodium methotrexate (aqueous & lyophilized). J. Pharm. Sci. Techno., 52(10): 23-27 (1998); Elorza, B., Elorza, M. A., Sainz, M. C., Chantres, J. R. Comparison of particle size and encapsulation parameters of three liposomal preparations. J. Microencapsul. 10(2): 237-248 (1993); Mykhaylyk, O., Sánchez-Antequera, Y., Vlaskou, D., Hammerschmid, E., Anton, M., Zelphati, O. and Plank, C. Liposomal Magnetofection. Methods Mol. Bio., 605: 487-525 (2010) (each of these publications is incorporated by reference in its entirety for all purposes)). Erythrocyte-mediated transfer is similar to liposome fusion and has been shown to have high efficiency and efficacy across all cell types tested (Microinjection and Organelle Transplantation Techniques; Celis et al. Eds.; Academic Press: New York, 1986 (which is incorporated by reference in its entirety for all purposes)). Typically erythrocytes are loaded by osmotic shock methods or electroporation methods (Schoen, P., Chonn, A., Cullis, P. R., Wilschut, J., and Schuerrer, P. Gene transfer mediated by fusion protein hemagglutinin reconstituted in cationic lipid vesicles. Gene Therapy 6: 823-832 (1999); Li, L. H., Hensen, M. L., Zhao, Y. L., Hui, S. W. Electrofusion between heterogeneous-sized mammalian cells in a pellet: potential applications in drug delivery and hybridoma formation. Biophysical Journal 71:479-486 (1996); Carruthers, A., & Melchior, D. L. A rapid method of reconstituting human erythrocyte sugar transport proteins. Biochem. 23: 2712-2718 (1984) (each of these three publications is incorporated by reference in its entirety for all purposes). Alternatively, erythrocytes may be loaded indirectly by loading hematopoietic progenitors with single-celled organisms and inducing them to differentiate and expand into erythrocytes containing single-celled organisms.

In an embodiment, natural means are used to introduce the artificial endosymbiont into host cells. The artificial endosymbiont of the invention can be introduced into host cells by a number of methods known to those of skill in the art including, but not limited to natural phagocytosis, induced phagocytosis, macropinocytosis, other cellular uptake processes, and the like. (See Microinjection and Organelle Transplantation Techniques, Celis et al. Eds.; Academic Press: New York, 1986 and references therein, (incorporated by reference in its entirety for all purposes)). Naturally phagocytotic cells have been show to take up bacteria, including MTB (Burdette, D. L., Seemann, J., Orth, K. Vibrio VopQ induces PI3-kinase independent autophagy and antagonizes phagocytosis. Molecular microbiology 73: 639 (2009); Wiedemann, A., Linder, S., Grassi, G., Albert, M., Autenrieth, I., Aepfelbacher, M. Yersinia enterocolitica invasin triggers phagocytosis via β1 integrins, CDC42Hs and WASp in macrophages. Cellular Microbiology 3: 693 (2001); Hackam, D. J., Rotstein, O.D., Schreiber, A., Zhang, W., Grinstein, S. Rho is required for the initiation of calcium signaling and phagocytosis by Fcγ receptors in macrophages. J of Exp. Med. 186(6): 955-966 (1997); Matsunaga, T., Hashimoto, K., Nakamura, N., Nakamura, K., Hashimoto, S. Phagocytosis of bacterial magnetite by leucocytes. Applied Microbiology and Biotechnology 31(4): 401-405 (1989) (each of these publications is incorporated by reference in its entirety for all purposes)).

In an embodiment, a chemical means is used to assist introduction of the artificial endosymbiont into the host cell. In an embodiment, the host cells are treated with a chemical so that the host cell has an increased ability to acquire artificial endosymbionts. In an embodiment, the artificial endosymbionts are treated with chemical(s) so that the artificial endosymbiont has an increased ability to be introduced into host cell. In an embodiment, both the host cells and artificial endosymbionts are treated with chemical(s). Recent studies have shown that non-phagocytotic cell types can be induced to endocytose bacteria when co-cultured with various factors: media and chemical factors, biologic factors (e.g., baculovirus, protein factors, genetic knock-ins, etc.). (See, e.g., Salminen, M., Airenne, K. J., Rinnankoski, R., Reimari, J., Valilehto, O., Rinne, J., Suikkanen, S., Kukkonen, S., Yla-Herttuala, S., Kulomaa, M. S., Vihinen-Ranta, M. Improvement in nuclear entry and transgene expression of baculoviruses by disintegration of microtubules in human hepatocytes. J. Virol. 79(5): 2720-2728 (2005); Modalsli, K. R., Mikalsen, S., Bukholm, G., Degre, M. Microinjection of HEp-2 cells with coxsackie B1 virus RNA enhances invasiveness of Shigella flexneri only after prestimulation with UV-inactivated virus. APMIS 101: 602-606 (1993); Hayward, R. D. & Koronakis, V. Direct nucleation and bundling of actin by the SipC protein of invasive Salmonella. The EMBO Journal 18: 4926-4934 (1999); Yoshida, S., Katayama, E., Kuwae, A., Mimuro, H., Suzuki, T., Sasakawa, C. Shigella deliver an effector protein to trigger host microtubule destabilization, which promotes Rac1 activity and efficient bacterial internalization. The EMBO Journal 21: 2923-2935 (2002); Bigildeev et al. J. Exp Hematol., 39: 187 (2011); Finlay, B. B. & Falkow, S. Common themes in microbial pathogenicity revisited. Microbiol. and Mol. Biol. Rev. 61: 136-169 (1997) (each of these publications is incorporated by reference in its entirety for all purposes)).

In an embodiment, chemical poration is used to assist in the introduction of artificial endosymbionts. In an embodiment, the host cell membranes are treated with permeabilization agents. In an embodiment, the permeabilization agents are selected from, for example, detergents (e.g., Trtiton X-100), digitonin, pore forming polypeptides (e.g., streptolysin O), saponin, and other agents well-known in the art to permeabilize host cell membranes. In an embodiment, hypotonic solutions are used in the chemical poration methods to induce flow of fluid and artificial endosymbionts into host cells. In an embodiment, hypotonic conditions are combined with cell permeabilization agents to assist in the introduction of artificial endosymbionts into host cells. References describing such chemical poration methods include, for example, Medepalli, K., Alphenaar, B. W., Keynton, R. S., Sethu, P., A new technique for reversible permeabilization of live cells for intracellular delivery of quantum dots, Nanotechnol. 24:205101 (2013); Hapala, I., Breaking the barrier: methods for reversible permeabilization of cellular membranes, Crit. Rev. Biotechnol. 17:105-122 (1997); Miyamoto, K., Yamashita, T., Tsukiyama, T., Kitamura, N., Minami, N., Yamada, M., Imai, H., Reversible membrane permeabilization of mammalian cells treated with digitonin and its use for inducing nuclear reprogramming by Xenopus egg extracts, Cloning Stem Cells 10:535-542 (2008); Johnson, J. A., Gray, M. A., Karliner, J. S., Chen, C-H., Mochly-Rosen, D., An improved permeabilization protocol for introduction of peptides into cardiac myocytes: application to protein Kinase C research, Circ. Res. 79:1086-1099 (1996), each of these publications is incorporated by reference in its entirety for all purposes.

In an embodiment, natural means also includes macropinocytosis or “cell drinking,” is a method numerous bacteria and viruses employ for intracellular entry (Zhang (2004) In: Molecular Imaging and Contrast Agent Database (MICAD) [database online]; Bethesda (Md.): National Library of Medicine (US), NCBI; 2004-2011 (each of these publications is incorporated by reference in its entirety for all purposes)). Various protocols exist which can be employed, to induce cells to take up bacteria. Several agents, such as nucleic acids, proteins, drugs and organelles have been encapsulated in liposomes and delivered to cells (Ben-Haim, N., Broz, P., Marsch, S., Meier, W., Hunziker, P. Cell-specific integration of artificial organelles based on functionalized polymer vesicles. Nano Lett. 8(5): 1368-1373 (2008); Lian, W., Chang, C., Chen, Y., Dan, R., Luo, Y., Chien, J., Hsieh, S., Lin, C. (each of these publications is incorporated by reference in its entirety for all purposes)).

In an embodiment, the means for enriching host cells with endosymbionts can be a selective agent, a selective media, a device that detects and sorts host cells using, a phenotype introduced by the artificial endosymbiont (e.g., a cell sorter), a chromatography column that separates host cells based on a phenotype imparted to the host cell by the artificial endosymbiont, or the like. In an embodiment, selective agents can be antibiotics or other agents that are toxic or inhibitory to the artificial endosymbiont or to the host cell. A selective agent that is toxic or inhibitory to artificial endosymbionts will kill or inhibit artificial endosymbionts that are exposed to the agent and artificial endosymbionts that are taken up by host cells can be protected from the agent by the host cell. A selective agent that is toxic or inhibitory to the host cell can be detoxified by the artificial endosymbiont (or modified artificial endosymbiont) and so host cells containing the artificial endosymbiont can be resistant to the selective agent. In an embodiment, a selective media is used that is missing a nutrient that the host cell needs to grow, and that the host cell cannot make. In this embodiment, the artificial endosymbiont (or modified artificial endosymbiont) provides the missing nutrient to the host cell and so the host cells with artificial endosymbionts can grow on the selective media.

In an embodiment, the artificial endosymbiont imparts a phenotype to the host cell that allows host cells with the artificial endosymbiont to be separated from host cells that lack the artificial endosymbiont. For example, if the artificial endosymbiont contains a florescent protein or a luciferase it can impart a light emitting phenotype to the host cell. This light emitting phenotype can be used to separate host cells with an artificial endosymbiont from host cells without an artificial endosymbiont using a cell sorter that detects and sorts based on emitted light. In an alternate example, the artificial endosymbiont is a magnetic bacterium that imparts to the host cell a magnetic phenotype. This magnetic phenotype can be used to enrich host cells with the artificial endosymbiont by a variety of means. For example, a magnetic field can be used to retard the flow of host cells with artificial endosymbionts or a magnetic field can be used to preferentially sediment host cells with artificial endosymbionts, or a magnetic field can be used to preferentially retain host cells with artificial endosymbionts. In an embodiment, a magnetic column is used to enrich host cells containing artificial endosymbionts that have imparted a magnetic phenotype to the host cell.

In an embodiment, the system has a means for detecting host cells with artificial endosymbionts. In an embodiment, the means for detecting can be a device that detects a phenotype imparted to the host cell by the artificial endosymbiont. The artificial endosymbiont may provide the host cell with a new property such as an optical property (e.g., light emission) or a magnetic property, or the artificial endosymbiont can change a property of the cell such as, expression of surface proteins, size, attachment ability, surface charge, weight, density, shape, resistance to selective agent, ability to metabolize a certain nutrient, or the like. Means for detecting these new or changed host cell properties are well-known in the art, and include, for example, optical devices that can detect light emitted from an artificial endosymbiont, or devices that can detect the magnetic properties of magnetosomes from an artificial endosymbiont such as MRI systems, magnetic particle imaging (MPI) systems, magnetic relaxation switching (MRS) systems, magnetic resonance spectrometers, superconducting quantum interference devices (SQUID), magnetometers, nuclear magnetic resonance (NMR) systems, and electron paramagnetic resonance (EPR) systems, and Mossbauer spectrometers, magnetic circular dichroism systems, Fluorescent activated cell sorter systems (FACS), and automated cell counters.

In an embodiment, the detection of artificial endosymbionts is done as a Quality Control (“QC”) and/or Quality Assurance (“QA”) step to measure the efficiency, concentration, and/or quantity of artificial endosymbiont introduced into the host cell. In an embodiment, the artificial endosymbiont is a magnetic bacteria and a magnetic property of the host cells with the magnetic bacteria is measured to quantify the efficiency, concentration, and/or quantity of magnetic bacteria (artificial endosymbiont) introduced into the host cell. In an embodiment, the artificial endosymbiont introduces an optical property into the host cell and the QC and/or QA step measures the amount of light or other optical property to measure the efficiency, concentration, and/or quantity of artificial endosymbiont introduced into the host cells. In an embodiment, an indirect measurement is made to quantify efficiency, concentration, and/or quantity of artificial endosymbiont introduced into the host cells. For example, the artificial endosymbiont may be measured using a reagent that reacts with the artificial endosymbiont inside the host cells. Or the system may measure the residual artificial endosymbiont remaining in the media as a measure of the artificial endosymbionts introduced into the host cells. In an embodiment, the reagent is specific for the artificial endosymbiont, e.g., an antibody. In an embodiment, the reagent interacts with a product made by the artificial endosymbiont that is secreted into the host cell. In an embodiment, the antibody is a polyclonal or monoclonal antibody made using a whole artificial endosymbiont or a specific antigen of the artificial endosymbiont. In an embodiment, the antibody recognizes an antigen or antigens on the surface of the artificial endosymbiont. In an embodiment, the antibody is a polyclonal rabbit antibody that recognizes surface antigens on AMB-1 (the rabbit is immunized with whole AMB-1). In an embodiment, the artificial endosymbiont is a magnetic bacteria (that may be modified as described above) and a polyclonal rabbit antibody is used to detect the magnetic bacteria in the eukaryotic host cells using techniques well-known in the art. (Immunocytochemical Methods and Protocols, Methods in Molecular Biology, Vol. 115 (ed. Lorene C. Javois) Jan. 15, 1999, which is incorporated by reference in its entirety for all purposes) For example, after magnetic bacteria have been introduced to the eukaryotic host cells, the host cells can be fixed and permeablized, allowing fluorescently labeled antibodies to enter the host cell to tag the magnetic bacteria. (Immunocytochemical Methods and Protocols, Methods in Molecular Biology, Vol. 115 (ed. Lorette C. Javois) Jan. 15, 1999, which is incorporated by reference in its entirety for all purposes) The strength of the signal obtained from the binding of the antibody can then be used to measure the magnetic bacteria in the host cells. Alternatively, the antibodies can be unlabeled, and detection of antibody bound to the magnetic bacteria is performed with a secondary antibody that recognizes the anti-magnetic bacteria antibodies. (Immunocytochemical Methods and Protocols, Methods in Molecular Biology, Vol. 115 (ed. Lorette C. Javois) Jan. 15, 1999, which is incorporated by reference in its entirety for all purposes) In an embodiment, the secondary antibody may be labeled with a reporter.

In an embodiment, the means for detecting measures a temperature and the thermal response of the host cells with artificial endosymbionts when placed in an alternating magnetic field is used to assess the artificial endosymbionts. In an embodiment, the alternating magnetic field has a frequency in the range of about 100 kHz to 500 kHz. The artificial endosymbionts (e.g., magnetic bacteria) absorb the energy from the alternating magnetic field and dissipate this energy as heat. In an embodiment, the release of heat can be measured and used to quantify the artificial endosymbionts in the host cells. In an embodiment, the systems of the invention perform this thermal measurement as a QA/QC step to insure that a suitable quantity of artificial endosymbionts reside within the host cell for an application.

In an embodiment, the means for detecting measures light scattering by the host cells. In an embodiment, the means for detecting measures light scattering when the host cells containing artificial endosymbionts, e.g., magnetic bacteria, are subjected to a magnetic field. In this embodiment, when host cells contain sufficient magnetic bacteria the host cells with the magnetic bacteria will orient in the magnetic field into either low light scattering or high light scattering orientations. This allows the quantification of host cells that have received sufficient artificial endosymbionts to allow orientation in a magnetic field. In an embodiment, the systems of the invention perform this light scattering detection measurement as a QA/QC step to ensure that a suitable quantity of artificial endosymbionts are contained within the host cells for an application.

In an embodiment, the means for detection measures a property (e.g., optical) of a reporter (e.g., GFP or luciferase) made by the artificial endosymbiont. In an embodiment, the signal from the reporter is measured and used to quantify the artificial endosymbionts in the host cells. In an embodiment, the reporter is constitutively expressed in the artificial endosymbiont. In an embodiment, the reporter is expressed by an inducible control region, such as those that are well-known in the art, and the system provides the inducing condition and then measures the reporter. In an embodiment, the systems of the invention perform this measurement of the reporter as a QA/QC step to insure that a suitable quantity of artificial endosymbionts reside within the host cell for an application.

In an embodiment, the QA/QC measurement compares the signal obtained to a standard curve made with a known quantity of reporter. In an embodiment, the systems of the invention perform the QA/QC measurement and utilizes the information from a standard curve to determine whether an appropriate quantity of artificial endosymbionts resides within the host cells.

### Kits and Methods for Detecting Artificial Endosymbionts in Host Cells

Kits of the invention include consumable items that will be used in the systems of the invention. The Kits comprise items of the system that are consumed when the system is used, or that need to be periodically replaced. Consumable items used in the system, include, for example, artificial endosymbionts, host cells, media, selective agents, containers, magnetic columns, tissue culture treated dishes, antibodies specific for the artificial endosymbiont, other reagents and accessories (e.g., containers) for use in detecting the artificial endosymbiont in the host cells, and the like. Items that will be periodically replaced include, for example, columns used in the system, needles for microinjection, or the like.

In an embodiment, kits of the invention include 1, 2, 3, 4, or more consumables used in a system of the invention. In an embodiment, the kit comprises artificial endosymbionts, or artificial endosymbionts with a media, or artificial endosymbionts, a media, and a selective agent. In an embodiment, the media of the kit is capable of killing or significantly disrupting bacteria, for example, the media may contain gentamycin. This way only endosymbionts inside the host cells will survive.

In an embodiment, the kit comprises a magnetic bacterium, an antibody specific for the magnetic bacteria, a magnet, a selective agent, and a reporter, or the kit may comprise any combination of the foregoing. The kit may also contain a magnetic column. In an embodiment, the magnetic bacteria of the kit are modified with a reporter. In an embodiment, the reporter is GFP. In an embodiment, the antibody in the kit is labeled with a fluorescent label. In an embodiment, the kit includes a secondary antibody for detecting the anti-magnetic bacteria antibody. In an embodiment, the selective agent is contained in the magnetic column. In an embodiment, the kit comprises a magnetic bacteria and a magnet.

The invention will be better understood from the experimental details which follow. However, one skilled in the art will readily appreciate that the specific methods and results discussed are merely illustrative of the invention as described more fully in the claims which follow thereafter.

### EXAMPLES

### Example 1

### Host Cells Containing an Artificial Endosymbiont with a Reporter

A. Construction of gfp⁺AMB.

Expression vectors for eGFP, one including a Shine-Dalgarno sequence upstream of the gfp gene and one without a Shine Dalgarno, sequence were cloned into cryptic broad host range vector pBBR1MCS-2 (Kovach, M. E., et al. Four new derivatives of the broad-host-range cloning vector pBBR1MCS, carrying different antibiotic-resistance cassettes. Gene 166, 175-176, (1995) (which is incorporated by reference in its entirety for all purposes)). AMB (ATCC 700264) was transformed with this construct. (Matsunaga, T. et al. Complete genome sequence of the facultative anaerobic magnetotactic bacterium Magnetospirillum sp. strain AMB-1. DNA Res. 12, 157-166 (2005); Burgess J. G., et al. Evolutionary relationships among Magnetospirillum strains inferred from phylogenetic analysis of 16S rDNA sequences. J. Bacteria 175: 6689-6694 (1993); Matsunaga T, et al. Gene transfer in magnetic bacteria: transposon mutagenesis and cloning of genomic DNA fragments required for magnetosome synthesis. J. Bacteriol. 174: 2748-2753 (1992); Kawaguchi R, et al. Phylogeny and 16s rRNA sequence of Magnetospirillum sp. AMB-1, an aerobic magnetic bacterium. Nucleic Acids Res. 20: 1140, (1992) (each of these four publications is incorporated by reference in its entirety for all purposes)).

Transformation was achieved by conjugation using a donor Escherichia coli strain as described by Goulian, M. van der Woude, M. A. A simple system for converting lacZ to gfp reporter fusions in diverse bacteria. Gene 372, 219-226 (2006); Scheffel, A. Schüler, D. The Acidic Repetitive Domain of the Magnetospirillum gryphiswaldense MamJ Protein Displays Hypervariability but Is Not Required for Magnetosome Chain Assembly. J Bacteriol. September; 189(17): 6437-6446 (2007) (each of these two publications is incorporated by reference in its entirety for all purposes). The mating reactions were cultured for 10 days under defined microaerophilic conditions in the absence of DAP to select for positive transformants.

Following conjugation, gfp⁺AMB transformants with and without the Shine-Dalgarno sequence successfully displayed GFP fluorescence. The transformants containing the Shine-Dalgarno sequence displayed higher levels of GFP fluorescence than the transformance without this sequence. The resulting fluorescence did not leave the gfp⁺AMB cells when viewed at 100× magnification at 488 nm excitation.

The magnetic properties of the gfp⁺AMB were analyzed by MRI. The gfp⁺AMB was suspended in agar plugs using a 1.5T instrument to optimize and characterize the imaging properties.

Cell visualization was tested in a mouse bearing two subcutaneous tumors, one on its left flank and one on its right flank. 1.5×10⁶ MDA-MB231 cells containing introduced gfp+AMB were injected directly into the tumor on the left flank of the mouse. An equivalent number of control MDA-MB231 cells without introduced cells were injected on the right flank of the mouse. The mouse was imaged using a bench top 1T MRI with T2w pulse sequences. The resulting image showed a dark area at the tumor on the right side of the mouse, the site of the injection of MDA-MB231 cells containing introduced gfp+AMB, but no signal at the tumor on the right side of the mouse, where control MDA-MB231 cells injected into a left side tumor.

B. Microinjection into Murine Embryonic Cells.

The gfp⁺AMB was microinjected into one cell of each of 170 mouse embryos at the 2-cell stage. Six concentrations over a log scale up to ˜10⁵ gfp⁺AMB were injected per cell, estimated by the optical density at 565 nm. Death rate of cells following microinjection was constant across the different injected concentrations. Images overlaying fluorescent and differential interference contrast (DIC) images of cells injected with the highest concentration (10⁵ MTB/cell) were compared. An uninjected control exhibited low levels of autofluorescence. Slices at different horizontal planes in 8-cell embryos at a given time point were compared. In each embryo, all four cells derived from the injected cell showed significant fluorescence while none of the four cells derived from the uninjected internal controls displayed significant fluorescence.

The embryos were allowed to develop for three days after the injection. In each concentration level, embryos survived for up to the full three days developing to the 256 cell blastula stage and appeared healthy enough for implantation. Numerous cells within each blastula displayed significant fluorescence, demonstrating that the artificial endosymbionts were transferred to daughter cells across multiple cell divisions as the embryos comprising the eukaryotic host cells developed to the blastula stage.

Confocal microscopy was used to quantify total expression of GFP throughout four individual embryos by measuring total GFP fluorescence in the entire embryo over time at various points beginning at the eight cell stage of the embryo. This indicates that the copy number of artificial endosymbionts was maintained in daughter cells for at least seven generations, such that the fluorescent phenotype of the host cells was maintained as the embryo progressed from the 2-cell stage to the 256-cell blastula stage.

These results demonstrate that, when delivered by microinjection, gfp⁺AMB were not immediately cleared or degraded and were not toxic to the developing embryo over the course of the three day experiment. Microinjected embryos divided normally, suggesting that gfp⁺AMB do not display pathogenic markers or secret toxic compounds. They were transferred to daughter cells across many cell divisions, were contained in the cytoplasm, were punctate and well distributed, and maintained copy number within the daughter host cells, such that the fluorescent phenotype of the eukaryote host cells was maintained in daughter cells through at least seven generations. These results demonstrate that AMB can be stably maintained intracellularly and are transferred to daughter cells over at least seven cell divisions.

### Example 2

### A Magic System for Making Host Cells with Artificial Endosymbionts

In this embodiment, a magnetic system is used to make host cells containing magnetic bacteria. The system comprises a modified artificial endosymbiont, host cells, a NdFeB, Grade N52 magnet, a tissue culture dish, host cell media with and without a selective agent, a magnetic column with a selective agent, and a FACS sorter.

An artificial endosymbiont, Magnetospirillum magneticum AMB-1 (ATCC® 700264™) is modified by mating it with an Escherichia coli strain BW29437 that contains a transferable plasmid with Kanamycin resistance and carrying eGFP in an expression cassette. Kanamycin positive, M. magneticum AMB-1 colonies are isolated and GFP expression is verified by fluorescent microscopy.

gfp⁺ AMB-1 are treated with Listeriolysin O protein (LLO) by placing the gfp⁺ AMB-1 into a solution of 0.6 μM LLO. The gfp⁺ AMB-1 are incubated in the LLO solution for 10-30 minutes at room temperature, and then the gfp⁺ AMB-1 cells are washed with PBS.

The host cells are breast cancer cell line MDA-MB231. A 6-well tissue culture dish and an NdFeB. Grade N52 magnet are used to assist in the introduction of modified M. magneticum gfp⁺ AMB-1 coated with LLO into the MDA-MB231 host cells. The media is DMEM supplemented with 10% Fetal Calf Serum. The media can also contain gentamycin or streptomycin as a selective agent. A MACS® magnetic column (Miltenyi Biotec) containing or incubated with gentamycin or streptomycin is used in the system to separate MDA-MB231 host cells with modified gfp⁺ M. magneticum AMB-1 coated with LLO from MDA-MB231 host cells without the artificial endosymbiont. A Fluorescence Activated Cell Sorter (FACS) is used to confirm the presence of artificial endosymbionts in the MDA-MB231 host cells, and to separate MDA-MB231 host cells with fluorescent artificial endosymbionts from host cells lacking fluorescent artificial endosymbionts.

MDA-MB231 host cells are grown in 6-well tissue culture dish to 40-70% confluence and overlaid with 1.5×10¹⁰ gfp⁺M. magneticum AMB-1 coated with LLO which are resuspended in 1 ml DMEM media supplemented with 10% Fetal Calf Serum. The dish is then mounted on the NdFeB, Grade N52 magnet. The cells are incubated on the magnet for 3 hours. After this incubation the cells are placed into media with gentamycin or streptomycin. Following 48 hours incubation to remove non-incorporated bacteria the host cells are trypsinized and passed through a magnetic column. The host cells with artificial endosymbionts are isolated from the magnetic column by elution with the growth media following the removal of the magnet. The isolated host cells are passed through a FACS sorter. The FACS sorter enriches for MDA-MB231 host cells that contain fluorescent artificial endosymbionts.

These isolated host cells with artificial endosymbionts may be used for any number of downstream applications.

### Example 3

### An Acceleration System for Making Host Cells with Artificial Endosymbionts

In this embodiment, an acceleration system is used to make host cells containing magnetic bacteria. The system comprises a modified artificial endosymbiont, host cells, a centrifuge, a tissue culture dish, host cell media with and without a selective agent, a magnetic column, and a FACS sorter.

An artificial endosymbiont, Magnetospirillum magneticum AMB-1 (ATCC® 700264™) is modified by mating it with an Escherichia coli strain BW29347 that contains a transferable plasmid with Kanamycin resistance and carrying eGFP in an expression cassette. Kanamycin positive, gfp⁺ M. magneticum AMB-1 colonies are isolated and GFP expression is verified by fluorescent microscopy.

The host cells are MDA-MB231 cells. A 6-well tissue culture dish and Sorvall Legend RT or similar table-top centrifuge are used to assist in the introduction of gfp⁺ M. magneticum AMB-1 into the MDA-MB231 host cells. The media is DMEM supplemented with 10% Fetal Calf Serum. The media can also contain gentamycin or streptomycin as a selective agent. A MACS® magnetic column (Miltenyi Biotec) is used in the system to separate MDA-MB231 host cells with gfp⁺ M. magneticum AMB-1 from MDA-MB231 host cells without the artificial endosymbiont. A FACS sorter is used to confirm the presence of artificial endosymbionts in the MDA-MB231 host cells, and to separate MDA-MB231 host cells with fluorescent artificial endosymbionts from host cells lacking fluorescent artificial endosymbionts.

MDA-MB231 host cells are grown in 6-well tissue culture dish to 40-70% confluence and overlaid with 1.5×10¹⁰ gfp⁺ M. magneticum AMB-1 resuspended in 1 ml DMEM media supplemented with 10% Fetal Calf Serum. The dish is then mounted on the Sorvall Legend RT centrifuge. The cells are spun in the centrifuge at 2000 rpm for 10 minutes, and then incubated for 3 hours. After this incubation the cells are placed into media with gentamycin or streptomycin, and after 48-hr incubation passed through a magnetic column. The host cells with artificial endosymbionts are isolated from the magnetic column by elution with the growth media following the removal of the magnet. The isolated host cells are then passed through a FACS sorter. The FACS sorting enriches for MDA-MB231 host cells that contain fluorescent artificial endosymbionts.

These isolated host cells with artificial endosymbionts may be used for any number of downstream applications.

### Example 4

### A Multiwell, Magnetic System for Making Host Cells with Artificial Endosymbionts

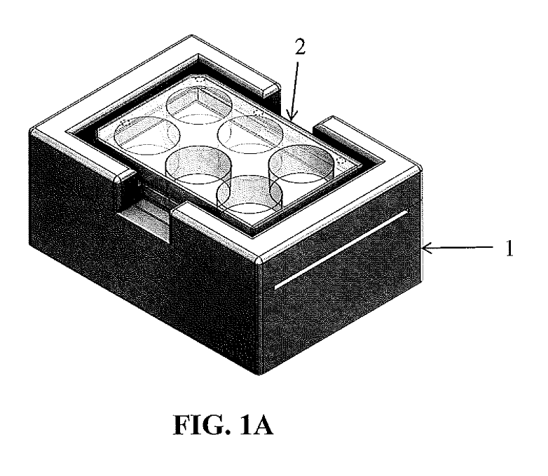

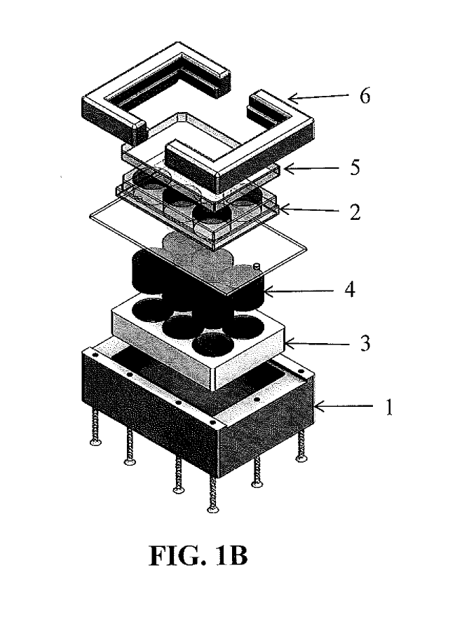

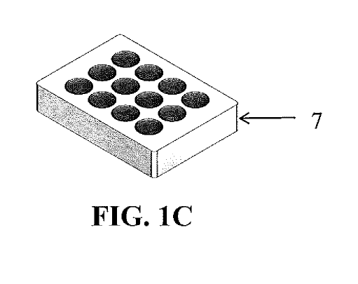

FIG. 1A depicts a system utilizing a magnetic means for introducing artificial endosymbionts into host cells. The system comprises a large base (1) with a multiwell container (2) for holding eukaryotic host cells and artificial endosymbionts (e.g., magnetic bacteria).

FIG. 1B depicts an exploded view of the system with the large base (1), a plate with multiple containers (or wells) (2), interchangeable insert (3) for holding the magnetic means (4), a top for the multiwell plate (5), and bumpers (6) for orienting the multiwell plate.

FIG. 1C depicts an alternative format insert (7) for holding twelve magnetic means (instead of six) for use with a twelve well plate.

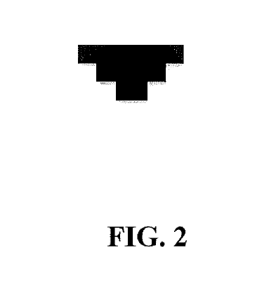

FIG. 2 depicts a magnetic means which is a NdFeB permanent magnet made with three layers, where the top layer is largest and each subsequent layer has less area that the preceding layer. The top layer of the NdFeB permanent magnet has a diameter that is larger than the diameter of the container to be used with the magnetic means. The design for the magnetic means produces a magnetic field strength that is similar across an individual well of the multiwell plate. In this embodiment, the interchangeable insert may be made to mate with the magnetic means (i.e., each cavity has steps that match the layers of the magnetic means). In an embodiment, the magnetic means is secured to the cavity in the interchangeable insert. In an embodiment, the magnetic means is secured to the cavity by an adhesive.

When the system is used to introduce artificial endosymbionts the following steps may be used. Host cells are introduced into wells of a six well plate. The host cells adhere to the surface of the wells and/or are suspended in the media. The host cells can be subject to a growth phase prior to introduction of artificial endosymbionts, e.g., MTB. Artificial endosymbionts (MTB) are added to the wells containing host cells, and the six well plate is placed into the system of FIG. 1A. The host cells and MTB are incubated together over the magnetic means for 1-16 hours, and then the plate is removed from the system. MTB which have not entered host cells are removed by washing the host cells. Optionally, host cells with MTB are separated from host cells without MTB (or with insufficient MTB to produce a desired phenotype) by passing the host cells with and without MTB over a magnetic column whereby the host cells with MTB pass more slowly through the column as the introduced MTB interact with the magnetic column to impede the flow of the cells in which they reside. Host cells with MTB are isolated and can then be used in any number of downstream applications.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

We claim:

1. A system comprising a magnetic bacteria, a mammalian cell, a container, and a means for introducing the magnetic bacteria into the mammalian cell, wherein the magnetic bacteria and the mammalian cell are inside the container.

2. The systems of claim 1, wherein the magnetic bacteria are modified to have a reporter.

3. The system of claim 2, wherein the reporter is a luciferase protein or a fluorescent protein.

4. The system of claim 3, wherein the reporter is a fluorescent protein.

5. The system of claim 1, further comprising a selective agent wherein the selective agent is inside the container.

6. The system of claim 5, wherein the selective agent is selected from the group consisting of a gentamycin, a kanamycin, and a chloramphenicol.

7. The system of claim 1, further comprising an anti-magnetic-bacteria antibody.

8. The system of claim 7, wherein the anti-magnetic-bacteria antibody is labeled with the reporter.

9. The system of claim 7, further comprising a secondary antibody specific for the anti-magnetic-bacteria antibody, and wherein the anti-magnetic-bacteria antibody is a polyclonal antibody or a monoclonal antibody.

10. The system of claim 1, wherein the means for introducing subjects the magnetic bacteria and the mammalian host cell to a magnetic field.

11. The system of claim 10, wherein the means for introducing is a permanent magnet.

12. A system comprising a magnetic bacteria, a mammalian cell, a container, a means for introducing the magnetic bacteria into the mammalian cell, and a means for detecting the mammalian cell containing the magnetic bacteria, wherein the magnetic bacteria and the mammalian cell are inside the container.

13. The system of claim 12, wherein the means for detecting is an optical device.

14. The systems of claim 12, wherein the magnetic bacteria is modified to have a reporter.

15. The system of claim 14, wherein the reporter is a luciferase protein or a fluorescent protein.

16. The system of claim 15, wherein the reporter is a green fluorescent protein.

17. The system of claim 12, further comprising an anti-magnetic-bacteria antibody.

18. The system of claim 17, wherein the anti-magnetic-bacteria antibody is labeled with a reporter.

19. The system of claim 18, further comprising a secondary antibody specific for the anti-magnetic-bacteria antibody, and wherein the anti-magnetic-bacteria antibody is a polyclonal antibody or a monoclonal antibody.

20. A system comprising a magnetic bacteria, a mammalian cell, a container, an anti-magnetic-bacteria antibody, and a means for introducing the magnetic bacteria into the mammalian cell, wherein the magnetic bacteria is genetically modified to express a gene encoding a green fluorescent protein, and wherein the modified magnetic bacteria, the mammalian cell, and the anti-magnetic-bacteria antibody are inside the container.
